(19) United States
(12) Patent Application Publication
(10) Pub. No.: US 2017/0007848 A1
(43) Pub. Date: Jan. 12, 2017

(54) Particle beam treatment system with solenoid magnets

(71) Applicants: Jürgen Drees, Wuppertal (DE); Helmut Piel, Wuppertal (DE)

(72) Inventors: Jürgen Drees, Wuppertal (DE); Helmut Piel, Wuppertal (DE)

(73) Assignee: Cryoelectra GmbH, Wuppertal (DE)

(21) Appl. No.: 15/203,966

(22) Filed: Jul. 7, 2016

(30) Foreign Application Priority Data
Jul. 8, 2015 (DE) 10 2015 111 060.1

(51) Int. Cl. A61N 5/10; H01J 37/147; H01J 37/14

(52) U.S. Cl. CPC A61N 5/10; H01J 37/14; H01J 37/1472; A61N 2005/1087; H01J 2237/141; H01J 2237/152

(57) ABSTRACT

A particle beam treatment system having a beam generation unit for generating a beam of charged particles, in particular ions, preferably protons, and having a beam guidance system. The generic beam guidance system takes up less space but can provide comparable or even improved beam properties because, in part, the beam guidance system seen in the direction of the beam of charged particles and behind the beam generation unit has at least one solenoid magnet as a beam shaping unit, and the at least one solenoid magnet of the beam guidance system is a superconducting solenoid magnet.

### CROSS-REFERENCE TO RELATED PATENT APPLICATION

This patent application claims priority to German Application No. 10 2015 111 060.1, filed Jul. 8, 2015, the entire teachings and disclosure of which are incorporated herein by reference thereto.

### FIELD OF THE INVENTION

According to a first aspect, the invention concerns a particle beam treatment system with a beam generation unit for generating a beam of charged particles, in particular ions, preferably protons, and with a beam guidance system. According to a second aspect, the invention also concerns an advantageous method.

### BACKGROUND OF THE INVENTION

In the state of the art, the abovementioned particle beam treatment systems are used for radiotherapy with charged particles, for example ions in the form of protons. Compared with the hitherto usual radiotherapy with ion, in particular photons there are considerable advantages for patients with certain cancers. Irradiation with protons is particularly advantageous, because these only achieve their maximum ionisation strength, and thus their associated destructive power, for example in relation to tumour cells, at the end of their path through the tissue to be irradiated, at what is known as the Bragg peak. In this way the damage to healthy tissue upstream of the tissue to be treated in the path of the beam can be reduced. In addition, the damage to healthy tissue downstream of the beam is almost fully avoided.

Successful treatment of tumours with a particle beam, for example using the raster-scan therapeutic procedure, however, calls for a particle beam with precise beam properties, in particular of the beam position or of the beam momentum, so that at the treatment site (known as the isocentre) treatment of the tissue can take place precisely and with no treatment inaccuracies, so that as far as possible no undesired irradiation of healthy tissue (for instance, adjacent organs) occurs.

The prior art particle beam treatment systems, that is to say the systems as a whole and in particular the beam guidance systems, however, take up a comparatively large amount of space. In addition, the beam properties and the transmission of the particle beam u to the treatment site that can be achieved are in need of improvement.

From WO 2009/106603 A1, for example, a particle beam treatment system is known, wherein a particle beam is generated and via a beam guidance system fed to one of several treatment rooms. It is proposed, in order to reduce operating costs, to provide different beam guidance systems, tailored to certain beam properties. Through the use of a single beam generation unit for multiple treatment locations this can indeed save space. But, this is only possible where several treatment locations are provided for and a lot of space is available any way. In addition, the beam guidance system and the switches provided for therein require a comparatively large amount of room.

From EP 2 268 359 B1 a particle beam therapy system is also known, wherein to guarantee precise beam guidance it is proposed, by means of a beam position monitor to monitor the beam properties during treatment in the halo region of the particle beam. While this allows better checking of the beam properties, the beam properties are still limited by the device technology, meaning that there is still a need to improve the beam properties. Such a system also requires a comparatively large amount of space.

Against this background, the object of the present invention is to propose a generic beam guidance system, which takes up less space but can provide comparable or even improved beam properties. A further problem for the invention is to propose an advantageous particle beam treatment system and an advantageous method.

### BRIEF SUMMARY OF THE INVENTION

In a generic particle beam treatment system, the problem is solved according to a first aspect, in that the beam guidance system seen in the direction of the beam of charged particles behind the beam generation unit has at least one solenoid magnet as a beam shaping unit, and the at least one solenoid magnet of the beam guidance system is a superconducting solenoid magnet.

It has been shown that by the provision of one or more solenoid magnets as beam shaping units in the beam guidance system, wherein the one solenoid magnet or the several solenoid magnets is/are superconducting, firstly improved beam properties, for example increased transmission up to the treatment site, and secondly a reduction in the space required by the particle beam treatment system, can be achieved. By means of the at least one solenoid magnet in particular the phase space of the beam of charged particles can be optimally prepared to continue. The at least one solenoid magnet works like a converging lens and reduces the divergence of the beam of charged particles. It has further been shown that the beam of charged particles also has good beam properties in terms of rotational symmetry. The combination of advantageous beam properties and reduced space requirements has previously not been achieved in the state of the art in this combination. It has, for example, been shown that it is possible to reduce the length of the particle beam treatment system measured up to the treatment site to less than 16 m, in particular less than 14 m.

The beam guidance system serves in particular for transporting the beam of charged particles with certain properties up to the treatment site. This means that the beam guidance system can in particular serve for shaping the beam of charged particles and for deflecting the beam of charged particles. Similarly, the beam guidance system can serve to check the properties of the beam of charged particles, for example if beam monitors are provided in the beam guidance system. Similarly, the beam guidance system can provide or allow screening of the treatment site from the beam generation unit, for example if a drift distance is provided which allows the provision of a screen, for example in the form of a concrete wall.

The beam generation unit is, for example, an accelerator, for example an accelerator which generates a beam of charged particles with constant energy, for example a cyclotron. It is also conceivable, however, for the beam generation unit to be an accelerator, which generates a beam of charged particles with variably adjustable energy, for example a synchrotron. Preferably a beam generation unit is used, emitting charged particles with an (average) kinetic energy of more than 200 MeV, for example between 200 MeV and 300 MeV, in particular between 210 MeV and 250 MeV. Preferably a beam generation unit with a diameter of less than 4 m is used, in order to keep down the space required by the particle beam treatment system.

The beam of particles is preferably an ion beam, in particular a proton beam. The beam guidance system is for example designed for targeted guidance of the beam of charged particles with energies of 60 to 210 MeV. 1 MeV corresponds to around 1.6×10⁻¹³ Joules. Here particle energy means in particular the average or maximum kinetic energy of the particles.

A solenoid magnet means in particular a cylindrical coil for generating a (as far as possible spatially constant) magnetic field. Here the beam of charged particles is guided through the inside of the solenoid magnet. Here the magnetic field runs through the inside of the solenoid magnet, in particular substantially parallel to the beam of charged particles. For example, the conductor windings (for example the wire windings) of the solenoid magnet are positioned on a cylindrical surface and are thin compared with the cylinder diameter.

Superconducting solenoid magnets means in particular that the magnetic field is at least partially generated by a flow of current through a superconducting material, that is to say a superconductor. A superconductor means a material with an electrical resistance that (suddenly) drops to zero once the temperature falls below a transition temperature. In this way high currents can be passed through the solenoid, allowing high magnetic fields and thus effective beam shaping.

Examples of superconductors that can be used here are NbTi or Nb₃SN.

The fact that the at least one solenoid magnet is provided as a beam shaping unit, means that a property of the beam of charged particles can be influenced by the at least one solenoid magnet. For example, the divergence of the beam of charged particles can be reduced. In particular, by means of the at least one solenoid magnet the beam of charged particles is not deflected as for example by a beam deflection unit, rather the beam of charged particles on average continues to move in the same direction.

The fact that at least one solenoid magnet is provided, means that preferably also more than one superconducting solenoid magnet can be provided. Here the solenoid magnets can be designed to be different from or identical to one another.

According to a preferred embodiment of the particle beam treatment system according to the invention at least one solenoid magnet of the beam guidance system is provided directly behind the beam generation unit. Since the at least one solenoid magnet is provided directly behind the beam generation unit, the beam properties can be influenced relatively early and focussing of the beam of charged particles on subsequent elements disposed in the beam can be achieved.

The fact that at least one solenoid magnet is provided directly behind the beam generation unit, means in particular that no other beam shaping units and/or beam deflection units are provided between the beam generation unit and the one solenoid magnet. It is conceivable, however, that for instance a beam monitor for monitoring the beam properties can also be provided between the beam generation unit and the one solenoid magnet.

According to a preferred embodiment of the particle beam treatment system according to the invention the beam guidance system has an energy correction unit for adjusting the energy of the charged particles of the beam of charged particles. Here at least one solenoid magnet of the beam guidance system seen in the direction of the beam of charged particles is provided between the beam generation unit and the energy correction unit and/or at least one solenoid magnet of the beam guidance system, seen in the direction of the beam of charged particles is provided behind the energy correction unit.

As a result of the energy correction unit, it is possible to flexibly adjust the energy of the charged particles of the beam of charged particles. It is possible, for example, to use a beam generation unit (for example a cyclotron), emitting charged particles with substantially constant (average) energy, without having to dispense with the ability to flexibly adjust the energy of the charged particles of the beam of charged particles. The energy correction unit is for example configured to reduce the energy of the charged particles of the beam of charged particles. The energy correction unit is for example a degrader. As a result, only one beam generation unit, emitting particles with sufficient maximum energy, needs to be provided. By means of the energy correction unit the energy of the charged particles of the beam of charged particles can then be adjusted as required.

The energy of the charged particles of the beam of charged particles can, for example, be reduced by reducing the (average) kinetic energy of the charged particles. As a result of the energy correction unit, decelerating of the charged particles to a selectable average kinetic energy can, for example, take place.

It has been shown that the design of the beam guidance system can be sufficiently compact that, for example, the treatment site can be positioned less than 10 m, in particular approximately 9 m away from the end of the energy correction unit.

A beam generation unit can, for example, be used emitting charged particles with a kinetic energy of more than 200 MeV, for example between 200 and 300 MeV, in particular between 210 and 250 MeV. The energy correction unit can, for example, be one by which the kinetic energy of the charged particles can be reduced, for example, to below 200 MeV and/or to below 100 MeV.

If at least one solenoid magnet of the beam guidance system seen in the direction of the beam of charged particles is provided between the beam generation unit and the energy correction unit, the transmission of the beam of charged particles up to the treatment centre can be increased. This can be attributed to the fact that mapping of the phase space emitted by the beam generation unit to the energy correction unit disposed (directly) behind can take place in such a way that as large as possible a part (in the optimum case all) of the phase space contains charged particles, which contribute to the transmission up to the treatment site. In other words, the beam of charged particles can advantageously be focused on or mapped to the energy correction unit. In this way a high proportion of the charged particles can pass the energy correction unit and contribute to a high transmission by the energy correction unit. Preferably exactly one solenoid magnet is provided between the beam generation unit and the energy correction unit.

Despite the provision of one solenoid magnet of the beam guidance system between the beam generation unit and the energy correction unit, the distance from the beam generation unit to the end of the energy correction unit can advantageously be less than 2 m.

If at least one solenoid magnet of the beam guidance system seen in the direction of the beam of charged particles is provided behind the energy correction unit, a divergence of the beam of charged particles after the energy correction unit can be countered and the beam of charged particles can advantageously be focused on or mapped to the subsequent elements disposed in the beam. In this way a high proportion of the charged particles can pass the beam guidance system up to the treatment centre and contribute to a high transmission by the beam guidance system. Preferably no other beam shaping units, beam deflection units and/or other magnetic units are provided between the energy correction unit and the one solenoid magnet. It is conceivable, however, for a collimator, for example, to be provided between the energy correction unit and the one solenoid magnet. Preferably exactly one solenoid magnet seen in the direction of the beam of charged particles is provided behind the energy correction unit.

Despite the provision of one solenoid magnet of the beam guidance system behind the energy correction unit the distance from the end of the energy correction unit to the treatment site can preferably be less than 10 m, in particular approximately 9 m.

The energy correction unit preferably has at least one block-shaped energy correction element moveable transversally to the beam of charged particles and at least one wedge-shaped energy correction element moveable transversally to the beam of charged particles.

A block-shaped energy correction element comprises, for example, an entry side and an exit side for the beam of charged particles, running substantially in parallel. A block-shaped energy correction element can for example be a cuboid.

A wedge-shaped energy correction element comprises, for example an entry side and an exit side for the beam of charged particles, running oblique, that is to say not in parallel, to one another.

Through the advantageous combination of at least one block-shaped and at least one wedge-shaped energy correction element, in particular, a compact and accurate reduction of the energy of the charged particles of the beam of charged particles can be achieved. As a result, in particular, of the compactness (i.e. a short distance to be traveled by the beam of charged particles) by means of such an energy correction unit excessive expansion of the phase space of the beam of charged particles can be avoided. The block-shaped energy correction element provides, for example, a first rough adjustment of the energy of the charged particles of the beam of charged particles. The energy of the charged particles of the beam of charged particles can, for example, be adjusted to discrete values. The wedge-shaped energy correction element provides, for example, compared with the first adjustment, a finer adjustment of the energy of the charged particles of the beam of charged particles. The energy of the charged particles of the beam of charged particles can, for example, be continuously adjusted (for example, within a certain range). By moving the wedge-shaped energy correction element transversally to the beam of charged particles, for example, the widening of the wedge-shaped energy correction element, seen in the direction of the beam of charged particles, in the area of the beam of charged particles is changed. By disposing the block-shaped energy correction elements, seen in the direction of the beam of charged particles, before the wedge-shaped energy correction elements, the latter can be designed to be correspondingly shorter, since the energy only needs to be adjusted over a smaller area.

The at least one block-shaped energy correction element, for example, is moveable in a direction (for example in the x-direction) transversally to the beam of charged particles. The at least one wedge-shaped energy correction element is, for example, moveable in the same direction and/or in the transversal direction perpendicular thereto (e.g. in the x-direction and/or in the y-direction) transversally to the beam of charged particles.

Preferably the at least one block-shaped energy correction element and the at least one wedge-shaped energy correction element are moveable substantially perpendicularly to the beam of charged particles.

Preferably the energy correction unit has a plurality of block-shaped energy correction elements moveable transversally to the beam of charged particles, allowing different adjustments of the energy of the beam of charged particles.

The plurality of (preferably different) block-shaped energy correction elements allows a flexible adjustment of the energy of the charged particles of the beam of charged particles over a wide energy range. The block-shaped energy correction elements can, for example, seen in the direction of the beam of charged particles, have different widenings and/or comprise different materials.

The energy correction unit preferably has several, in particular two transversally wedge-shaped energy correction elements moveable transversally to the beam of charged particles.

The energy correction unit is, for example, configured so that the wedge-shaped energy correction elements can be positioned simultaneously in the beam of charged particles. For example, one wedge-shaped energy correction element is moveable from a first direction into the beam of charged particles and another wedge-shaped energy correction element is moveable from a second direction (for example from an opposite direction to the first direction) into the beam of charged particles.

In this way, the range within which fine-tuning is possible is broadened. In addition, in this way an asymmetrical reduction of the energy over the transverse section of the beam of charged particles can be avoided. To this end, for example, two wedge-shaped energy correction elements are disposed mirror-symmetrically or point-symmetrically to one another.

The energy correction unit is preferably made at least in part of boron carbide. In contrast to the materials used in the state of the art, by using boron carbide (B₄C) a more compact and reliable energy correction unit can be provided. The high proportion of boron (atomic number 5) and the high density of boron carbide reduce the expansion of the phase space compared to the materials used previously. For example, compared to energy correction units made (exclusively) of graphite, an up to 30% more compact energy correction unit can be provided. The distance from the exit window of the beam generation unit (for example an accelerator) to the end of the energy correction unit seen in the direction of the beam of charged particles is preferably less than 2 m, in particular less than 1.5 m. At the same time, less widening of the energy correction unit in the direction of the beam of charged particles can result in less expansion of the beam of charged particles. Ultimately this allows improved beam properties and improved transmission properties of the particle beam treatment system. Furthermore, compared to an energy correction unit containing beryllium an energy correction unit with lower toxicity can be provided.

It is similarly possible to provide an energy correction unit made of different materials, for example boron carbide and graphite. For example, the block-shaped and wedge-shaped energy correction elements can comprise different materials.

For example, inter alia though the use of such an energy correction unit in combination with superconducting solenoid magnets the beam of charged particles with an energy of 215 MeV can be decelerated to 70 MeV, wherein a transmission or more than 3% to the treatment site can still be achieved. With the use, for example, of at least one superconducting solenoid magnet and with a deceleration to 90 MeV, five times the transmission can be achieved than when, for example, just quadrupoles are used. Compared to values from the state of the art therefore, significantly higher transmission values can be achieved.

The beam guidance system seen in the direction of the beam of charged particles preferably has a collimator unit after the energy correction unit. This allows the phase space after the energy correction unit to be restricted and the beam quality at the treatment site to be improved. The collimator unit is preferably disposed directly after the energy correction unit. The collimator unit is, for example, designed as a screen. A screen is, for example, a block of material with one or more openings. Each of the screens, for example, has a circular and/or seen in the opposite direction to the beam of charged particles tapered opening. The beam of charged particles passes through the collimator unit preferably in a vacuum.

As already stated, the collimator unit is preferably disposed between the energy correction unit and a solenoid magnet provided directly behind.

According to a preferred embodiment of the particle beam treatment system according to the invention the beam guidance system has at least at least two solenoid magnets seen in the direction of the beam of charged particles behind the beam generation unit.

By providing at least two solenoid magnets the beam properties can be further improved with greater compactness of the particle beam treatment system. It has proven particularly advantageous that the provision of exactly two solenoid magnets in the beam guidance system is advantageous. In this way advantageous beam properties can be achieved with a low space requirement and less complicated systems engineering.

As already stated, it is particularly advantageous if a first solenoid magnet is provided between the beam generation unit and the energy correction unit and a second solenoid magnet is provided behind the energy correction unit, in particular between the energy correction unit and a gantry.

According to a preferred embodiment of the particle beam treatment system according to the invention at least one solenoid magnet is designed as a cylindrical coil running in a substantially linear direction. If a number of solenoid magnets are provided, preferably each of these solenoid magnets is designed as a cylindrical coil running in a substantially linear direction. In this way simply built solenoid magnets can be provided, which can be used as beam shaping units. The cylindrical coil, for example, has a single layer design. In particular, the conductor (for example a wire) of the cylindrical coil can have a spiral or helical path.

According to a preferred embodiment of the particle beam treatment system according to the invention the windings of the at least one solenoid magnet run substantially perpendicularly to the beam of charged particles. If several solenoid magnets are provided, this preferably applies to all solenoid magnets. This means in particular that the windings of the conductor of the respective solenoid magnet in each case run substantially in a plane perpendicular to the beam of charged particles. The windings are thus not tilted in relation to the beam of charged particles.

According to a preferred embodiment of the particle beam treatment system according to the invention the at least one solenoid magnet generates at least in sections a substantially homogenous magnetic field. A homogenous magnetic field means that the field strength is the same at each point within the solenoid magnet, and thus spatially homogenous. Here the substantially homogenous magnetic field is generated in the area which the beam of charged particles crosses and which in this regard is critical to the forming of the beam of charged particles. If several solenoid magnets are provided, this preferably applies to all solenoid magnets.

According to a preferred embodiment of the particle beam treatment system according to the invention, the magnetic field of at least one solenoid magnet is in the range of 1 tesla to 10 tesla, preferably in the range of 4 tesla to 8 tesla and/or the magnetic field of at least one solenoid magnet is in the range of 5 tesla to 20 tesla, preferably in the range of 8 tesla to 15 tesla. Such magnetic field strengths allow effective shaping of the beam of charged particles and enable a high transmission up to the treatment centre. It is particularly advantageous if at least two solenoid magnets are provided and the magnetic fields of the solenoid magnets are different. The magnetic field of one solenoid magnet (for example of the first solenoid magnet seen in the beam direction, for example of the solenoid magnet disposed between the beam generation unit and the energy correction unit) is for example in the range of 5 tesla to 20 tesla, preferably in the range of 8 tesla to 15 tesla (for example approximately 11-11.5 tesla) and the magnetic field of the other solenoid magnet (for example the second solenoid magnet seen in the beam direction, for example the solenoid magnet disposed behind the energy correction unit) is in the range of 1 tesla to 10 tesla, preferably in the range of 4 tesla to 8 tesla (for example approximately 6-6.5 tesla).

According to a preferred embodiment of the particle beam treatment system according to the invention the particle beam treatment system is configured so that at least one solenoid magnet in the case of differing energies of the charged particles of the beam of charged particles generates a substantially constant magnetic field. This can in particular have the advantage that the field of the solenoid magnets (unlike quadrupole magnets, for example) does not have to be fully or partially controlled or regulated as a function of the energy of the energy of the charged particles, simplifying the design of the particle beam treatment system. The solenoid magnet, for example, independently of the energy of the charged particles generates a substantially constant magnetic field. The solenoid magnet, for example, for all or some of the adjustable particle energies (for example from 70 MeV to 200 MeV) generates a substantially constant magnetic field. If several solenoid magnets are provided, preferably all solenoid magnets in the case of differing energies of the charged particles of the beam of charged particles generate a substantially constant magnetic field. Here the different solenoid magnets can generate magnetic fields of different sizes.

According to a preferred embodiment of the particle beam treatment system according to the invention at least one solenoid magnet has an entry opening and/or exit opening of between 10 mm and 50 mm, preferably of between 20 mm and 40 mm, and/or at least one solenoid magnet has an entry opening and/or exit opening of between 30 mm and 70 mm, preferably of between 40 mm and 60 mm. It has been shown that with such entry openings or exit openings a desired beam shaping can be achieved. At least two solenoid magnets are, are for example, provided having different entry openings and exit openings. A first solenoid magnet, for example (for example the first solenoid magnet seen in the direction of the beam, for example the solenoid magnet disposed between the beam generation unit and the energy correction unit) has an entry opening and/or exit opening of between 10 mm and 50 mm, preferably of between 20 mm and 40 mm (for example of approximately 30 mm) and the other solenoid magnet (for example the second solenoid magnet seen in the direction of the beam, for example the solenoid magnet disposed behind the energy correction unit) an entry opening and/or exit opening of between 30 mm and 70 mm, preferably of between 40 mm and 60 mm (for example of approximately 52 mm).

According to a preferred embodiment of the particle beam treatment system according to the invention, the beam guidance system has an immovable section and a movable, in particular rotatable, section.

It has been shown that by providing at least one superconducting solenoid magnet as the beam shaping unit a high proportion of the charged particles can be guided into the rotatable section and the beam of charged particles allows a substantially rotationally symmetrical profile about the beam axis upon entering the rotatable section.

In order to create the movable section, the beam guidance system has, for example, a moveable supporting frame known as a gantry. The supporting frame can in an advantageous manner, be rotatable in particular about a horizontal axis, by up to 360°, in order to be able to irradiate the treatment site from as many angles as possible. The axis of rotation of the supporting frame coincides in particular with the original axis of the beam of charged particles (i.e. in particular with the axis of the beam of charged particles before deflection by a first beam deflection unit).

The immovable section of the beam guidance system is, for example, disposed between the beam generation device and the movable section of the beam guidance system.

The movable section (in particular the gantry) can in particular comprise further elements of the beam guidance system, in particular one or more (for example four) beam deflection units, one or more (for example seven) beam shaping units (for example in the form of quadrupole magnets), one or more (for example two) collimator units, one or more scanning magnets and/or one or more beam monitors.

It is basically also conceivable to design the movable section of the beam guidance system without collimator units. In this way the beam guidance system can have a more compact design.

The movable section of the beam guidance system can for example be designed so that the beam of charged particles is guided to the treatment site so that the beam of charged particles runs a maximum of 3 m away from the original axis of the beam of charged particles (for example how the beam leaves the beam generation unit).

According to a preferred embodiment of the particle beam treatment system according to the invention the at least one solenoid magnet is provided in the immovable section of the beam guidance system. If more than one solenoid magnet is provided (for example two) preferably all solenoid magnets are provided in the immovable section of the beam guidance system.

At least one (preferably all) solenoid magnet(s) is, for example, disposed between the beam generation unit and the rotatable section of the beam guidance system. A solenoid magnet is, for example, provided between the energy correction unit and the movable section of the beam guidance system. The moveable section of the beam guidance system preferably is free of solenoid magnets.

The energy correction unit (preferably including the collimator unit disposed behind it) is preferably provided in the immovable section of the beam guidance system.

According to a preferred embodiment of the particle beam treatment system according to the invention, the beam guidance system has at least one magnetic beam deflection unit, in particular in the movable section of the beam guidance system. If several magnetic beam deflection units are provided, preferably all are provided in the movable section of the beam guidance system. By means of the at least one beam deflection unit the beam of charged particles can advantageously and as needed be guided from the beam generation unit to the treatment centre. Here the magnetic beam deflection unit can have an entry side for entry of the beam of charged particles from any entry direction into the magnetic beam deflection unit and an exit side for emergence of the beam of charged particles in an exit direction from the magnetic beam deflection unit.

The at least one magnetic beam deflection unit is preferably a dipole magnet. As a result of the design of the magnetic beam deflection unit as a dipole magnet a substantially homogenous magnetic field can be provided in a simple manner for deflecting the beam of charged particles. In addition, dipole magnets are comparatively easy to manufacture. The dipole magnet is for example an electromagnet. The dipole magnet has, for example, an iron core, for example an iron yoke. The iron core can have iron plates, for example. The iron core is, for example, made of iron plates stacked on top of each other. This allows the dipole magnet to be manufactured in a simple manner. The dipole magnet can, for example, seen in the direction of the beam of charged particles, have a distance between the entry side and the exit side of 0.5-2 m, preferably approximately 1 m. It has been shown that the used of dipole magnets in the beam guidance system allows good beam properties.

The beam guidance system preferably has several, for example two, three or in particular four magnetic beam deflection units. If several magnetic beam deflection units are provided, a more complex path of the beam of charged particles in a compact beam guidance system can be achieved.

The several beam deflection units are preferably provided in the beam guidance system so that the beam of charged particles (at a certain point in time) runs substantially in one plane. By rotating the movable section, for example, this plane can be rotated.

The beam guidance system is, for example, configured so that the beam of charged particles is directed by the first beam deflection unit away from the original axis of the beam of charged particles and runs oblique to the original axis. The original axis of the beam of charged particles is, for example, the axis along which the beam of charged particles emerges from the beam generation unit and/or along which it moves before the first beam deflection unit. The beam guidance system is, for example, configured so that the beam of charged particles is directed by the further magnetic beam deflection units (for example a second, third and fourth magnetic beam deflection unit) back towards the original axis of the beam of charged particles. The beam guidance system is, for example, configured so that the beam of charged particles after the second beam deflection unit runs parallel to the original axis of the beam of charged particles. The beam guidance system is, for example, configured so that the beam of charged particles after the last (for example the fourth) beam deflection unit runs transversally, in particular substantially perpendicularly, to the original axis of the beam of charged particles and preferably crosses the original axis.

At least some of the several magnetic beam deflection units preferably have an identical design. In this way the cost of producing the magnetic beam deflection units can be reduced. All but one of the magnetic beam deflection units (for example the last magnetic beam deflection unit before the treatment site) can, for example, have an identical design. The fourth beam deflection unit, for example, has a different design. As a result of a differing design in particular of the last magnetic beam deflection unit it is possible to adapt to the conditions in the beam path. The fourth beam deflection unit, for example, compared with the other magnetic beam deflection units, has an enlarged entry opening and/or exit opening, in order to provide irradiation of a sufficient volume at the treatment site.

The second and third magnetic beam deflection units, seen in the direction of the beam of charged particles, for example, are 0.5 m to 2 m, preferably 1 m to 1.5 m apart. The last magnetic beam deflection unit seen in the direction of the beam of charged particles is a maximum of 1.5 m, preferably a maximum of 0.99 m away from the treatment site. The distance between two magnetic beam deflection units is in particular understood to be the distance from the exit point of the beam of charged particles from one magnetic beam deflection unit to the entry point of the beam of charged particles into the other magnetic beam deflection unit.

The beam of charged particles can, with the beam guidance system, for example, over a distance of less than 10 m, in particular less than 8 m, be directed away from the direction along the original axis of the beam of charged particles in a direction perpendicular to this and crossing the original axis.

The entry side of the (at least one) magnetic beam deflection unit is preferably, at least in sections, designed to be substantially parallel to the exit side.

It has been shown that, if a beam of charged particles is to be deflected away from an entry direction in an exit direction deviating from the entry direction, as a result of such as design of the beam deflection unit the beam properties and the compactness of the beam guidance system can be further improved. This is attributed, inter alia, to the fact that such magnetic beam deflection units can achieve not only a deflection of the beam of charged particles, but also a focusing of the beam of charged particles similar to a beam shaping unit. For the magnetic-optical properties of a magnetic beam deflection unit (for example a dipole magnet) are substantially determined by the angle of the incoming beam of charged particles to the entry and exit edge. As a result, less beam shaping units are necessary in the beam guidance system as a whole. In addition, increased transmission of the beam of charged particles by the beam guidance system can be achieved. This means that overall a more compact particle beam treatment system with improved beam properties can be provided. Furthermore, production of the beam deflection units, in which the entry side is designed at least in sections to be substantially parallel to the exit side, is also easier, meaning that manufacturing costs are also reduced.

The entry side and/or the exit side of the magnetic beam deflection unit, for example, has/have at least in sections a flat design. In this case, in particular the flat sections of the entry side and the exit side are at least in sections designed to be substantially parallel to one another. The entry side can in particular be an end surface, for example a front face of the beam deflection unit, for example having an entry opening for entry of the beam of charged particles. The exit side can in particular be a further end surface, for example an end surface opposite the entry side, for example a rear side of the deflection unit, which for example has an exit opening for exiting of the beam of charged particles.

Preferably the entire entry side and the entire exit side are substantially parallel to one another. Substantially parallel means that the entry side and the exit side, for example, enclose an angle of less than 5°, preferably an angle of less than 3°, particularly preferably of less than 1°.

The magnetic beam deflection unit is provided in the beam guidance system to deflect the beam of charged particles, for example, so that the beam is deflected from the entry direction into the desired exit direction.

The magnetic beam unit is preferably designed for constant deflection over time of the beam of charged particles. This means that the beam of charged particles is for example continuously deflected by 45°.

The beam deflection unit can for example be controllable, so that a desired deflection for example for different energies of the charged particles of the beam of the charged particles is possible.

The magnetic beam deflection unit can be provided in the beam guidance system to deflect the beam of charged particles so that the entry side is oblique to the entry direction of the beam of charged particles and/or the exit side is oblique to the exit direction of the beam of charged particles.

In the beam guidance system, the beam of charged particles can enter the beam deflection unit in particular from a defined entry direction and exit the beam deflection unit from a defined exit direction. Because the entry side is oblique to the entry direction of the beam of charged particles or the exit side is oblique to the exit direction of the beam of charged particles, the beam deflection unit can in particular be provided symmetrically in the beam of charged particles, further improving the beam properties. Here an oblique arrangement means in particular that one side is in particular not perpendicular and/or not parallel to the entry or exit direction. The entry and/or exit side, for example, run substantially parallel to the bisector of the angle formed between the entry direction and the exit direction of the beam of charged particles. The angle between the entry side and the entry direction, for example, and between the exit side and the exit direction is the same.

The magnetic beam deflection unit can be provided in the beam guidance system to deflect the beam of charged particles so that the entry direction and the exit direction are at an angle of 30° to 60°, preferably of 40° to 50°, in particular substantially 45°, to one another.

The beam of charged particles is thus, for example, deflected by 30° to 60°, preferably by 40° to 50°, in particular by substantially 45°. It has been shown that a deflection of the beam of charged particles at such angles can take place with good beam properties and at the same time a compact beam guidance system can be provided.

As already stated, in particular several such magnetic beam deflection units can be provided.

According to a preferred embodiment of the particle beam treatment system according to the invention at least one solenoid magnet seen in the direction of the beam of charged particles is disposed before the at least one magnetic beam deflection unit. As a result of disposing the solenoid magnet before a magnetic beam deflection unit the beam of charged particles is focused before the deflection. This can contribute to increasing the transmission up to the treatment centre. In particular, at least one solenoid magnet can be provided before the first magnetic beam deflection unit, so that the beam properties can be optimised before a first deflection of the beam of charged particles.

According to a preferred embodiment of the particle beam treatment system according to the invention the beam guidance system has at least one additional magnetic beam shaping unit, in particular in the form of a quadrupole magnet, in particular in the movable section of the beam guidance system.

As a result of providing magnetic beam shaping units, which are provided in addition to the at least one solenoid magnet, in particular in the form of quadrupole magnets, the beam property can be further improved and the beam of charged particles efficiently focused. It has been shown that in the beam guidance system comparatively few additional beam shaping units are necessary, to achieve good beam properties, allowing a compact beam guidance system. Generally, though, other types of additional beam shaping units can also be provided. For example, beam shaping units in the form of sextupole magnets can be provided.

The magnetic field of the additional magnetic beam shaping unit(s) (for example in the case of quadrupole magnets) is for example partially scaled (for example for energies of a maximum of 120 MeV) as a function of the average momentum of the particles of the beam of charged particles (after the energy correction unit). For greater energies a deviating scaling can be provided for.

Thanks to their design, quadrupole magnets can only focus a beam of charged particles in a direction transversal to the beam of charged particles. In this regard it is advantageous to provide at least two quadrupole magnets, in order to achieve beam shaping in both directions (i.e. in the plane) transversal to the beam of charged particles.

A minimum of five and/or a maximum of ten, preferably seven additional beam shaping units can, for example, be provided in the beam guidance system. A minimum of four and/or a maximum of six, preferably five additional beam shaping units are, for example, provided between a (seen in the direction of the beam of charged particles) first and a second magnetic beam deflection unit. Two additional beam shaping units are, for example, provided between the second and the third magnetic beam deflection units. Further additional beam shaping units can also be provided, however. Some, preferably all, of the additional beam shaping units, for example, have the same dimensions. This reduces the manufacturing costs of the beam guidance system.

The beam guidance system seen in the direction of the beam of charged particles preferably has a collimator unit before the magnetic beam deflection unit.

This allows the beam properties to be improved while taking up little additional space. As a result of the collimator unit, the phase space of the beam of charged particles of the beam further transported behind the collimator unit can be restricted.

The collimator unit is, for example, designed as a screen, for example as a block of material with one or more openings. The collimator unit, for example, has an angular, for example a rectangular opening. The opening is preferably variable in both directions transversally to the beam of charged particles. In this way a flexible tailoring of the beam properties can take place.

The beam guidance system preferably has at least two magnetic beam deflection units and between two of the at least two magnetic beam deflection units a collimator unit.

It has been shown that in particular between two beam deflection units a large momentum dispersion can dominate. By providing a collimator unit the beam properties can be further improved while taking up little additional space. It has been shown in particular that between the second and third beam deflection units a comparatively large momentum dispersion dominates, so that by providing a collimator unit the beam properties can be particularly improved. The collimator unit can therefore in particular be used to achieve a momentum selection for the beam of charged particles at the treatment site. In particular, shaping for the beam spot at the treatment site can take place. It is also possible to achieve losses after the collimator and up to the treatment site of less than 1%, although in the beam guidance system seen further in the beam direction further, for example two more beam deflection units are disposed.

The beam guidance system, before the first magnetic beam deflection unit seen in the direction of the beam of charged particles, preferably has a drift distance, with no magnetic beam deflection units and/or magnetic beam shaping units.

As a result of the drift distance advantageously an area can be provided, which for example can accommodate one or more measuring devices (in particular beam monitors) for beam control. Similarly, this area can serve to shield (for example by means of a concrete shield) the treatment site from the beam generation unit. The length of the drift distance is, for example, a minimum of 1 m and/or a maximum of 2 m. The drift distance is preferably at least partially provided in a vacuum, for example in a vacuum tube of preferably a few centimetres in diameter. The drift distance seen in the direction of the beam of charged particles is preferably disposed behind a solenoid magnet. The drift distance preferably has no magnetic components. The drift distance is preferably provided in the immovable section of the beam guidance system.

The particle beam treatment system and in particular the beam guidance system can have further units not mentioned here. A scanning magnet can, for example, be provided in the beam guidance system. The scanning magnet is, for example, provided between two beam deflection units. The scanning magnet is preferably provided between the last and the penultimate (for example between the third and fourth) beam deflection units, since in this position the scanning magnet advantageously allows a broad scanning range at the treatment site.

As a further example of a further unit, the beam guidance system can have one or more beam monitors. As a result of the beam monitors the beam properties, for example the beam position and the particle momentum of the beam of charged particles can in particular be measured at different points.

According to a second aspect of the present invention the abovementioned problem is also solved by a method, carried out with a particle beam treatment system according to the invention, comprising the steps of generating a beam of charged particles, in particular ions, preferably protons, with the beam generation unit, and guiding the beam of charged particles by means of the beam guidance system.

Regarding the advantages and further embodiments of the method, reference is made to the above aspect and its embodiments.

In particular, the above and the following description of means for carrying out a method step shall serve to disclose the corresponding method step. Similarly, disclosure of method steps shall also serve to disclose corresponding means or facilities for carrying out the method steps.

The examples and exemplary embodiments of all aspects of the present invention described above are intended also to be understood in all combinations with one another.

Further advantageous exemplary embodiments of the various aspects are indicated by the following detailed description of a number of exemplary embodiments of the aspects, in particular in combination with the figures. The figures accompanying the application, however, serve only for clarification and not to determine the scope of protection of the invention. The attached drawings are not necessarily to scale and are intended merely to illustrate examples of the general concept of the presents aspects. In particular, features contained in the figures should in no way be considered to be an essential component of the present invention.

### BRIEF DESCRIPTION OF THE SEVERAL VIEWS OF THE DRAWING

The figures show as follows:

FIG. 1 shows a schematic sectional view of an embodiment of a particle beam treatment system with an embodiment of a beam guidance system according to the present invention;

FIG. 2 shows a schematic sectional view of a further embodiment of the movable section of a beam guidance system;

FIG. 3 shows a schematic sectional view of an embodiment of an energy correction unit.

### DETAILED DESCRIPTION OF THE INVENTION

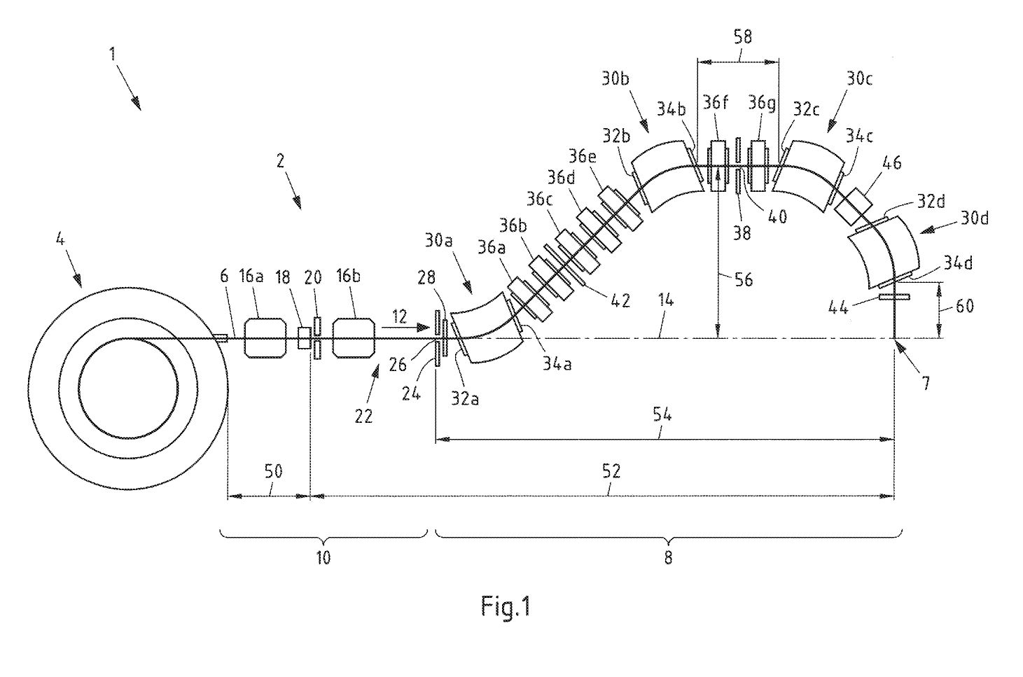

FIG. 1 shows a schematic sectional view of an embodiment of a particle beam treatment system 1 with an embodiment of a beam guidance systems 2. The particle beam treatment system comprises a beam generation unit 4 for generating a beam of charged particles 6, which in particular can be protons. The beam of charged particles 6 is guided by the beam guidance system 2 to a treatment site 7. The beam guidance system 2 has a section 8 that is moveable, in this case rotatable through 360°, and a section 10 that is immovable. The rotatable section 8 can for example be built as a supporting frame in the form of a gantry (not shown).

The beam generation unit 4 is in this case a cyclotron, that is to say an accelerator device which generates the beam of charged particles 6 with constant energy. The beam generation unit 4 emits charged particles with a constant kinetic energy, for example 210 MeV, 215 MeV or 250 MeV. It has been shown that by means of charged particles with a kinetic energy of approximately 207 MeV approximately 95%, and by means of charged particles with a kinetic energy of approximately 198 MeV still approximately 90%, of the patients to be treated can be treated.

The beam of charged particles runs initially in the direction of the arrow 12 along an original axis 14 of the beam of charged particles 6.

In the direction 12 of the beam of charged particles 6 seen behind the beam generation unit 4 a first superconducting solenoid magnet 16a is provided as a beam shaping unit. In this case the solenoid magnet 16a is provided directly behind the beam generation unit. Similarly, the solenoid magnet 16a is provided between the beam generation unit 4 and an energy correction unit 18. The magnetic field of the solenoid magnets 16a is in this case approximately 11.2 tesla. The entry opening and the exit opening of the solenoid magnet 16a are in this case approximately 30 mm. Depending on the construction of the particle beam treatment system and of the beam guidance system other magnetic field strengths and opening sizes may, however, be possible or necessary.

As a result of the solenoid magnet 16a improved mapping of the phase space emitted by the beam generation unit to the energy correction unit 18 disposed behind can be brought about, so that the beam quality and transmission of the beam of charged particles 6 up to the treatment site 7 can be increased.

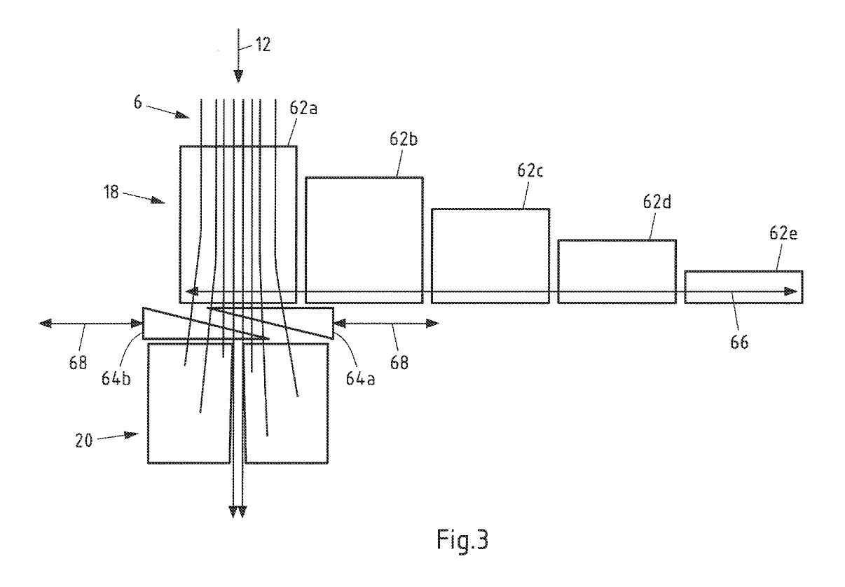

The beam of charged particles 6 is then guided through the energy correction unit 18. The energy correction unit 18 allows an adjustment of the energy of the charged particles of the beam of charged particles 6. The energy correction unit is described in more detail in connection with FIG. 3.

The beam guidance system 2 also has, seen in the direction 12 of the beam of charged particles 6, directly after the energy correction unit 18 a collimator unit 20, which the beam of the charged particles 6 then passes through. The collimator unit comprises a screen, which takes the form of a circular block of material, and seen opposite to the direction 12 of the beam of charged particles 6, respectively, a tapered opening.

Seen further in the direction of 12 of the beam of charged particles 6 behind the energy correction unit 18 and the collimator unit 20 a second superconducting solenoid magnet 16b is provided as a beam shaping unit. The solenoid magnet 16b is provided between the energy correction unit 18 and the movable section 8 of the beam guidance system 2 and in particular before a first magnetic beam deflection unit 30a. The magnetic field of the solenoid magnet 16b is in this case approximately 6.16 tesla. The entry opening and the exit opening of the solenoid magnet 16b are in this case approximately 52 mm. Depending on the construction of the particle beam treatment system and of the beam guidance system other magnetic field strengths and opening sizes may, however, be possible or necessary.

As a result of the solenoid magnet 16b improved mapping of the phase space emitted by the energy correction unit 18 to a collimator unit 24 disposed behind (and thus in the movable section 8 of the beam guidance system 2) can be brought about, so that the beam quality and transmission of the beam of charged particles 6 up to the treatment site 7 can be increased.

Both the first solenoid magnet 16a and the second solenoid magnet 16b, for example, take the form of a cylindrical coil running in a linear direction and generate in the area of the beam of charged particles 6 a substantially homogenous magnetic field. The particle beam treatment system 1 is advantageously configured so that the solenoid magnets 16a, 16b independently of the energy of the charged particles of the beam of charged particles 6 generate a substantially constant magnetic field, so that complex energy-based control of the solenoid magnet 16a, 16b can be dispensed with.

After the second solenoid magnet 16b seen in the direction of 12 of the beam of charged particles 6, the beam guidance system 2 has a drift distance 22. The drift distance 22 has no magnetic units, such as magnetic beam deflection units or magnetic beam shaping units. In the area of the drift distance 22 a shield, for example a concrete shield (not shown) can be provided. In addition, in the area of the drift distance 22 measuring devices such as beam monitors (not shown) can be provided.

The beam of charged particles 6 can pass through the section of the energy correction unit 18, the collimator unit 20 and/or the drift distance 22 of the beam guidance system 2, in a vacuum, which improves the beam properties and the transmission in this section.

The elements of the beam guidance system 2 described above are disposed in the immovable section 10 of the beam guidance system 2. To be able to irradiate the treatment site 7 from as many angles as possible, the rotatable section 8 is provided. The axis of rotation of the supporting frame (not shown) coincides with the original axis 14 of the beam of charged particles 6.

In the rotatable section 8 the beam guidance system first has a collimator unit 24. As a result of the collimator unit 24 the phase space of the beam of charged particles can be defined. The collimator unit 24 is designed here as a screen, in this case as a block of material with a rectangular opening 26. The geometry of the opening 26 is variable in both directions transversally to the beam of charged particles 6, to allow flexible tailoring of the beam properties.

Then an optional beam monitor 28 is provided, in order to check the beam properties. Additionally, or alternatively, the beam monitor can also be provided at other points of the beam guidance system 2.

Then, for guiding the beam of charged particles 6, the beam guidance system 2 has several, in this case four, magnetic beam deflection units 30a, 30b, 30c, 30d designed as magnetic dipoles. The magnetic beam deflection units 30a, 30b, 30c, 30d each have an entry side 32a, 32b, 32c, 32d for entry of the beam of charged particles 6 from an entry direction into the respective magnetic beam deflection unit. The magnetic beam deflection units 30a, 30b, 30c, 30d also each have an exit side 34a, 34b, 34c, 34d for the emergence of the beam of charged particles 6 in an exit direction from the magnetic beam deflection unit 30a, 30b, 30c, 30d. The entry sides 32a, 32b, 32c, 32d are in each case designed to be parallel to the respective exit side 34a, 34b, 34c, 34d. Here the magnetic beam deflection units 30a, 30b, 30c, 30d are in each case provided for deflecting the beam of charged particles 6 in the beam guidance system 2, so that the respective entry side 32a, 32b, 32c, 32d lies oblique to the respective entry direction of the beam of charged particles 6 and the respective exit side 34a, 34b, 34c, 34d oblique to the respective exit direction of the beam of charged particles 6. Here the magnetic beam deflection units 30a, 30b, 30c, 30d are in this case provided for deflecting the beam of charged particles 6 in the beam guidance system 2, so that the entry direction and the exit direction at each beam deflection unit 30a, 30b, 30c, 30d are at an angle of 45° to each other. The beam deflection units 30a, 30b, 30c, 30d here are positioned symmetrically in the beam of charged particles, that is to say that the angle between entry direction and entry side 32a, 32b, 32c, 32d and the angle between exit direction and exit side 34a, 34b, 34c, 34d are in each case identical for the individual beam deflection units 30a, 30b, 30c, 30d.

The beam of charged particles 6 is deflected by the first beam deflection unit 30a away from the original axis 14 of the beam of charged particles. The beam of charged particles 6 is deflected by the second beam deflection unit 30b back towards the original axis 14 and then runs parallel to the original axis 14 of the beam of charged particles 6. Then the beam of charged particles 6 is deflected by the third and the fourth beam deflection units 30c, 30d similarly back towards the original axis 14, so that the beam of charged particles 6 after the last beam deflection unit 30d is running perpendicularly to the original axis 14 of the beam of charged particles 6 and crosses the original axis 14.

As a result of the described development of the beam deflection units 30a, 30b, 30c, 30d improved beam properties can be achieved with a simultaneously more compact beam guidance system 2. This is attributed, inter alia, to the fact that the magnetic beam deflection units 30a, 30b, 30c, 30d can not only achieve a deflection of the beam of charged particles, but also a focusing of the beam of charged particles similar to a quadrupole magnet.

Here the magnetic beam deflection units 30a, 30b, 30c, 30d provided have a defocusing in a transversal direction (here, in the plane of projection) and a focusing in a direction perpendicular thereto. At this point the magnetic beam deflection units 30a, 30b, 30c, 30d have similar properties to quadrupole magnets, which similarly focus in a transversal direction and defocus in the direction perpendicular thereto. All four magnetic beam deflection units 30a, 30b, 30c, 30d therefore focus in just one direction (perpendicularly to the plane of projection). Of the seven quadrupole magnets 36a-36g provided (see below) therefore, five focus in the transversal direction in the plane of projection and only two in the direction perpendicular thereto. Thus overall sufficient focussing in both transversal coordinate directions is achieved. The result is that by focusing the dipoles in the y-direction therefore only two further quadrupoles with focusing in the same direction are necessary.

In addition, the manufacturing process for the beam deflection units 30a, 30b, 30c, 30d can be simplified as a result of the parallel entry and exit sides, since the iron core of the beam deflection units 30a, 30b, 30c, 30d can be made from parallel plates layered one on top of the other.

The beam guidance system 2 also has several, in this case seven, additional magnetic beam shaping units 36a, 36b, 36c, 36d, 36e, 36f, 36g in the form of quadrupole magnets. As a result of the beam shaping units 36a, 36b, 36c, 36d, 36e, 36f, 36g the beam property of the beam of charged particles 6 can be further improved. In particular, due, inter alia, to the solenoid magnets 16a, 16b and the beam deflection units 30a, 30b, 30c, 30d only a comparatively low number of additional beam shaping units 36a, 36b, 36c, 36d, 36e, 36f, 36g is necessary, to achieve good beam properties, allowing a compact beam guidance system 2.

Between the (seen in the direction 12 of the beam of charged particles 6) first magnetic beam deflection unit 30a and the second magnetic beam deflection unit 30b five additional beam shaping units 36a, 36b, 36c, 36d, 36e are provided. Between the second magnetic beam deflection unit 30b and the third magnetic beam deflection unit 30c a further two additional beam shaping units 36f, 36g are provided. The additional beam shaping units 36a, 36b, 36c, 36d, 36e, 36f, 36g in this case all have the same dimensions.

The beam guidance system 2 has, between the two magnetic beam deflection units 30b, 30c, a further collimator unit 38 in the form of a screen with a rectangular opening 40. The opening is variable in both directions transversal to the beam of charged particles 6, as a result of which shaping of the beam spot at the treatment site 7 can take place. It has been shown that between the beam deflection units 30b, 30c a comparatively large momentum dispersion dominates. This can be countered by providing the collimator unit 38, because the collimator unit 38 allows momentum selection for the beam of charged particles 6 at the treatment site 7.

In the beam guidance system 2 the beam monitors 42 and 44 are further provided. The beam monitor 42 is provided between the additional beam shaping units 36b and 36c. The beam monitor 44 is provided after the fourth beam deflection unit 30d and before the treatment centre 7.

Between the beam deflection unit 30c and the beam deflection unit 30d the beam guidance system also has a scanning magnet 46. The scanning magnet can be advantageously used at this position, since in this way at the treatment site a larger scanning range can be covered. With a system such as that previously proposed in 2005 by V. Anferov, for example, the beam can by way of example be moved so that at the treatment site an area of 210 mm×175 mm can be covered, with a deflection angle of just ±44 mrad in both coordinate directions. A further enlargement of the scanning range is possible. Since the beam deflection unit 30d must receive the charged particles 6 deflected by the scanning magnets 46, the beam deflection unit 30d can have a larger entry opening and/or exit opening than the other beam deflection units 30a, 30b, 30c. The beam deflection units 30a, 30b, 30c can be structurally identical.

Because the scanning magnet 46 is provided between the last and the penultimate (that is to say the third and fourth) beam deflection units 30c, 30d, this allows a larger scanning range at the treatment site 7.

It has been shown that a particularly compact beam guidance system 2 can be provided. The distance 50 from the beam generation unit 4 to the end of the energy correction unit 18 here is less than 2 m. The distance 52 from the end of the energy correction unit 18 to the treatment site 7 here is less than 10 m. Here the beam of charged particles 6 can be guided with the beam guidance system 2 over a distance 54 of less than 8 m from the direction 12 along the original axis 14 of the beam of charged particles 6 to the treatment site 7. Here the maximum distance 56 of the beam of charged particles 6 is less than 3 m from the original axis 14 of the beam of charged particles 6. Here the distance 58 of the second and third magnetic beam deflection units 30b, 30c is less than 15 m. Here the distance 60 of the last magnetic beam deflection unit 30d from the treatment site is less than 1 m, for example 0.991 m.

It is worth noting that the geometric dimensions refer to a beam of charged particles with a kinetic energy of approximately 210 MeV. If higher energies are used, the geometric dimensions are preferably multiplied by a factor. This geometric scaling factor is, for example, the precise ratio of the momentum of the higher energy protons (for example 245 MeV) to 210 MeV protons.

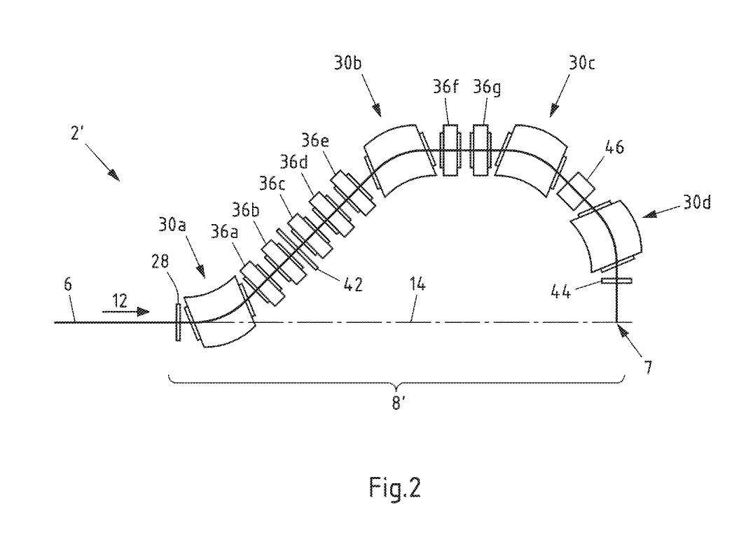

FIG. 2 is a schematic sectional view of a further embodiment of the movable section of a beam guidance system 2′. The rotatable section 8′ shown, of the beam guidance system 2′ from FIG. 2, is similar to the rotatable section 8 of the beam guidance system 2 from FIG. 1. In this regard, for the same elements the same reference signs are used. Equally, reference is made to the statements on the beam guidance system 2 from FIG. 1. In particular, the rotatable section 8′ can be provided instead of the rotatable section 8 in the beam guidance system 2. In the following only the differences from the beam guidance system 2 from FIG. 1 are considered.

The substantial difference between the beam guidance system 2 and the beam guidance system 2′ is that the beam guidance system 2′ does not have any collimator units 24, 38 in the rotatable section 8′. This means in particular that the distance from the magnetic beam deflection units 30b, 30c can be shortened, so that the distance of these can be less than 1.2 m, in particular less than 1.1 m. The phase space selection in this case has already taken place before the beam of charged particles 6 enters the rotatable section 8′.

FIG. 3 shows a schematic sectional view of an embodiment of an energy correction unit 18 in the form of a degrader, such as can, for example, be used in the beam guidance system 2 or 2′. The energy correction unit 18 has a plurality of block-shaped energy correction elements 62a, 62b, 62c, 62d, 62e moveable transversally to the beam of charged particles 6 and two wedge-shaped energy correction elements 64a, 64b moveable transversally to the beam of charged particles 6.

The block-shaped energy correction elements 62a, 62b, 62c, 62d, 62e are moveable here along the arrow 66 perpendicularly to the beam of charged particles 6. This allows differing adjustments of the energy of the charged particles of the beam of charged particles 6, depending on which of the block-shaped energy correction elements 62a, 62b, 62c, 62d, 62e is pushed into the beam of charged particles. For this, the block-shaped energy correction elements 62a, 62b, 62c, 62d, 62e seen in the direction 12 of the beam of charged particles 6, can have different widenings. Here the block-shaped energy correction elements 62a, 62b, 62c, 62d, 62e provide a rough adjustment of the energy of the charged particles of the beam of charged particles 6, because the energy of the charged particles of the beam of charged particles 6 can be adjusted to discrete values by the block-shaped energy correction elements 62a, 62b, 62c, 62d, 62e.

The wedge-shaped energy correction elements 64a, 64b are similarly movable along the arrow 68 similarly perpendicularly to the beam of charged particles 6. The wedge-shaped energy correction elements 64a, 64b provide fine-tuning of the energy of the charged particles of the beam of charged particles 6 after the beam of charged particles 6 has been guided through one of the block-shaped energy correction elements 62a, 62b, 62c, 62d, 62e. The energy of the charged particles of the beam of charged particles 6 can be continuously adjusted by the wedge-shaped energy correction elements 64a, 64b within a certain range. By moving the wedge-shaped energy correction elements transversally to the beam of charged particles the widening of the wedge-shaped energy correction element 64a, 64b seen in the direction of the beam of charged particles 6 can be changed in the area of the beam of charged particles. The two wedge-shaped energy correction elements 64a, 64b are in this case disposed point-symmetrically to one another. The angled surfaces of the wedge-shaped energy correction elements 64a, 64b are turned towards one another. This arrangement means that an asymmetrical reduction of the energy over the transverse section of the beam of charged particles 6 can be avoided.

The block-shaped energy correction elements 62a, 62b, 62c, 62d, 62e of the energy correction unit 18 are made of graphite and/or boron carbide. It is similarly conceivable, however, to provide block-shaped energy correction elements in different materials. The wedge-shaped energy correction elements 64a, 64b of the energy correction unit 18 are also made of graphite and/or of boron carbide. It is also conceivable here to provide wedge-shaped energy correction elements in different materials.

Due to the compactness of the energy correction unit 18 excessive expansion of the phase space of the beam of charged particles 6 can be avoided.

Seen in direction 12 of the beam of charged particles 6, behind the energy correction unit 18, a collimator 20 is provided. This can, for example, be the collimator shown in FIG. 1.

In summary, it is possible, using a smaller deflection angle, caused by the magnetic field of the scanning magnets 46, to achieve a large scanning range at the treatment site 7 and at the same time to keep down the distances of all magnetic elements from the axis of rotation 14.

The efficiency of the particle beam treatment system from FIG. 1 can be determined by means of more accurate Monte Carlo calculations. For example, the transmission efficiency at the treatment site 7 when decelerating an energy emitted by a cyclotron from 215 MeV to 70 MeV is still approximately 3.3%. This means that a cyclotron current of a few 10 nA is required in order to achieve a current of 1 nA at the treatment centre. For a deceleration to 90 MeV, the transmission when superconducting solenoid magnets are used is for example five times higher than when quadrupole magnets are used. The width of the beam spot here can be set at 3.6 mm (1σ) with a very low ellipticity (approximately 3-5%). The momentum width of the charged particles arriving at the treatment site 7 can be varied between 4 and 9 per thousand. At the same time, the size of the beam spot at the treatment site 7 is maintained. It has been shown that for a deceleration by the energy correction unit 18 to 90 MeV a transmission efficiency up to the treatment site of up to 10% can even be achieved.

For the above results it is assumed that the magnetic fields of the solenoid magnets are constant for particle energies of 70 MeV-200 MeV. The first solenoid 16a generates a magnetic field of 11.2 tesla with a cold bore of 30 mm. The second solenoid 16b generates a magnetic field of 6.16 tesla with a cold bore of 52 mm. The material of the energy correction unit 18 is assumed to be boron carbide. The magnetic fields of the quadrupole magnets scale for energies of 120 MeV with the average momentum of the decelerated beam.

The physical principles of the interaction between protons and matter and the programs used for calculating the properties of beam guidance systems are described in the following scientific reports:
Particle Data Group, W.-M. Yao et al., “The Review of Particle Physics”, Journal of Physics G33 (2006) 1 and update 2008.
Karl L. Brown, Sam K. Howry, “TRANSPORT, A Computer Program for Designing Charged Particle Beam Transport Systems”, SLAC Report No. 91 (1970), SLAC Report 91, Rev. 3 (1983) and later updates of the TRANSPORT program by U. Rohrer and others.
U. Rohrer, “PSI Graphic TURTLE Framework based on a CERN-SLAC-FERMILAB version by K. L. Brown et al.”, http://aea.web.psi.ch/Urs_Rohrer/MyWeb/turtle.htm.
J. Drees, “Passage of Protons through Thick Degraders”, Cryoelectra Report September 2008.
   The contents of these scientific reports are incorporated herein by reference in their entireties.

The principle of an x-y scanning magnet is described in the following scientific report:
V. Anferov, “Combined X-Y scanning magnet for conformal proton radiation therapy”, Med. Phys. 32 (3), March 2005.
   The contents of which are incorporated herein by reference in its entirety.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A particle beam treatment system comprising:
a beam generation unit for generating a beam of charged particles, in particular ions, preferably protons, and
a beam guidance system,
wherein the beam guidance system seen in the direction of the beam of charged particles behind the beam generation unit has at least one solenoid magnet as a beam shaping unit, and the at least one solenoid magnet of the beam guidance system is a superconducting solenoid magnet.

2. A particle beam treatment system according to claim 1,
wherein at least one solenoid magnet of the beam guidance system is provided directly behind the beam generation unit.

3. A particle beam treatment system according to claim 1,
wherein the beam guidance system has an energy correction unit for adjusting the energy of the charged particles of the beam of charged particles and at least one solenoid magnet of the beam guidance system seen in the direction of the beam of charged particles is provided between the beam generation unit and the energy correction unit and/or at least one solenoid magnet of the beam guidance system seen in the direction of the beam of charged particles is provided behind the energy correction unit.

4. A particle beam treatment system according to claim 1,
wherein the beam guidance system has at least two solenoid magnets seen in the direction of the beam of charged particles behind the beam generation unit.

5. A particle beam treatment system according to claim 1,
wherein at least one solenoid magnet is designed as a cylindrical coil running in a substantially linear direction.

6. A particle beam treatment system according to claim 1,
wherein the windings of at least one solenoid magnet run substantially perpendicularly to the beam of charged particles.

7. A particle beam treatment system according to claim 1,
wherein at least one solenoid magnet generates at least in sections a substantially homogenous magnetic field.

8. A particle beam treatment system according to claim 1,
wherein the magnetic field of a first solenoid magnet is in the range of 1 tesla to 10 tesla and the magnetic field of a second solenoid magnet is in the range of 5 tesla to 20 tesla.

9. A particle beam treatment system according to claim 1,
wherein the particle beam treatment system is configured so that at least one solenoid magnet in the case of differing energies of the charged particles of the beam of charged particles generates a substantially constant magnetic field.

10. A particle beam treatment system according to claim 1,
wherein a first solenoid magnet has an entry opening and/or exit opening of between 10 mm and 50 mm and a second solenoid magnet has an entry opening and/or exit opening of between 30 mm and 70 mm.

11. A particle beam treatment system according to claim 1,
wherein the beam guidance system has an immovable section and a movable section.

12. A particle beam treatment system according to claim 11,
wherein the at least one solenoid magnet is provided in the immovable section of the beam guidance system.

13. A particle beam treatment system according to claim 1,
wherein the beam guidance system has at least one magnetic beam deflection unit in the movable section of the beam guidance system.

14. A particle beam treatment system according to claim 13,
wherein at least one solenoid magnet seen in the direction of the beam of charged particles is disposed before the at least one magnetic beam deflection unit.

15. A particle beam treatment system according to claim 1,
wherein the beam guidance system has at least one additional magnetic beam shaping unit in the form of a quadrupole magnet.

16. A method, carried out with a particle beam treatment system according to claim 1, comprising the steps of:
generating a beam of charged particles with the beam generation unit, and
guiding the beam of charged particles by means of the beam guidance system.

17. A particle beam treatment system according to claim 8,
wherein the magnetic field of the first solenoid magnet is in the range of 4 tesla to 8 tesla and the magnetic field of a second solenoid magnet is in the range of 8 tesla to 15 tesla.

18. A particle beam treatment system according to claim 10,
wherein a first solenoid magnet has an entry opening and/or exit opening of between 20 mm and 40 mm and a second solenoid magnet has an entry opening and/or exit opening of between 40 mm and 60 mm.

19. A particle beam treatment system according to claim 11,
wherein the movable section is rotatable.

20. The method according to claim 16,
wherein the beam of charged particles includes protons.
